(19) United States
(12) Patent Application Publication
(10) Pub. No.: US 2012/0053128 A1
(43) Pub. Date: Mar. 1, 2012

(54) Method of inhibiting angiogenesis

(75) Inventors: Partha S. Bhattacharjee, Metairie, LA (US); Tarun Kumar Mandal, Kenner, LA (US)

(73) Assignee: XAVIER UNIVERSITY

(21) Appl. No.: 12/807,062

(22) Filed: Aug. 26, 2010

(51) Int. Cl. A61K 38/10; C12N 5/00; A61P 17/02; A61P 35/00

(52) U.S. Cl. 514/19.2; 435/375

(57) ABSTRACT

A method of inhibiting angiogenesis in mammals using introduction of an 18 amino acid tandem-repeat dimer peptide of apolipoprotein E residues 141-149 into the mammal, in an amount effective to inhibit angiogenesis in the mammal compared to the rate of angiogenesis which would occur in the mammal in the absence of the apolipoprotein E dimer peptide.

Part of this work was made using governmental support from the National Institutes of Health under grants EY019144, EY006311, and EY02377. The U.S. government has certain rights in this invention.

### BACKGROUND

Angiogenesis is the formation of new blood vessels from pre-existing vasculature. Angiogenesis is relevant not only to cancer but also to non-neoplastic diseases such as: macular degeneration, psoriasis, endometriosis, and arthritis. The growth and metastasis of tumors are dependent upon angiogenesis. Therefore, inhibiting angiogenesis can be used as a method of stopping tumor progression.

Human apolipoprotein E is involved in lipid metabolism and cardiovascular disorders; experimental studies of human apolipoprotein E have focused on its receptor binding region, located between residues 130-150. The receptor binding region contains a heparin-binding domain, residues 142-147, which mediates the attachment of apolipoprotein E to cellular heparin sulfate proteoglycan, an integral component of the extracellular matrix, which is involved in regulation of angiogenesis. Thus, there is a need for one or more methods of treatment having the ability to block these interactions.

Endothelial cells are the building blocks of angiogenesis. The interaction between vascular endothelial growth factor (VEGF), which is secreted by tumor cells, and endothelial cell vascular endothelial growth factor receptors, specifically VEGF receptor 2(Flk-1), initiates signaling pathways leading to angiogenesis, including angiogenesis in tumor cells. VEGF promotes endothelial cell survival, proliferation, and migration, mainly through the activation of the Flk-1 receptor.

Activated Flk-1 binds to c-Src and phosphorylates it. Subsequently, c-Src mediates phosphorylation of FAK. Both c-Src phosphorylation and c-Src mediated FAK phosphorylation are essential in angiogenesis. Activated c-Src is involved in activation of downstream ERK1/2 phosphorylation

Additionally, Akt phosphorylation can be a necessary antecedent for eNOS activation, endothelial cell migration, and, therefore, angiogenesis. VEGF-stimulated endothelial migration can result from increased nitric oxide production from eNOS phosphorylation.

### SUMMARY

An embodiment of the present invention is a method of inhibiting angiogenesis in mammals by introducing an 18 amino acid tandem-repeat dimer peptide of apolipoprotein E residues 141-149.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a graph illustrating the effect of apoEdp on HUVEC viability, as explained in Example 1.

FIG. 1B is a graph illustrating the effect of apoEdp on MDA-MB-231 cell viability, as explained in Example 1.

FIG. 2A is a representative photomicrograph of the VEGF control group described in Example 2.

FIG. 2B is a representative photomicrograph of the apoEdp treated test group described in Example 2.

FIG. 2C is a graph illustrating the effect of apoEdp on endothelial cell wound healing migration, as explained in Example 2.

FIG. 3A is a representative photomicrograph of the VEGF control group described in Example 3.

FIG. 3B is a representative photomicrograph of the apoEdp treated test group described in Example 3.

FIG. 3C is a graph illustrating the effect of apoEdp on endothelial cell capillary tubule formation, as explained in Example 3.

FIG. 4A is a representative photomicrograph of the VEGF control group described in Example 4.

FIG. 4B is a representative photomicrograph of the apoEdp treated test group described in Example 4.

FIG. 4C is a graph illustrating the effect of apoEdp on endothelial cell migration/invasion, as explained in Example 4.

FIG. 5A is a representative photograph of the saline treated control group described in Example 5.

FIG. 5B is a representative photograph of the apoEdp treated test group described in Example 5.

FIG. 5C is a graph illustrating the effect of apoEdp on angiogenesis, as explained in Example 5.

FIG. 6A is a representative photograph of the PBS treated control group described in Example 6.

FIG. 6B is a representative photograph of the apoEdp treated test group described in Example 6.

FIG. 6C is a graph illustrating the effect of apoEdp on tumor growth as explained in Example 6.

FIG. 7A is a graphical representation of the effect of apoEdp on VEGF-induced phosphorylation of Flk-1, as explained in Example 7.

FIG. 7B is a chemiluminescence photograph of the western blot analysis on the effect of apoEdp on VEGF-induced phosphorylation of Flk-1, as explained in Example 7.

FIG. 8A is a graphical representation of the effect of apoEdp on VEGF-induced phosphorylation of c-Src, as explained in Example 7.

FIG. 8B is a chemiluminescence photograph of the western blot analysis on the effect of apoEdp on VEGF-induced phosphorylation of c-Src, as explained in Example 7.

FIG. 9A is a graphical representation of the effect of apoEdp on VEGF-induced phosphorylation of Akt, as explained in Example 7.

FIG. 9B is a chemiluminescence photograph of the western blot analysis on the effect of apoEdp on VEGF-induced phosphorylation of Akt, as explained in Example 7.

FIG. 10A is a graphical representation of the effect of apoEdp on VEGF-induced phosphorylation of eNOS, as explained in Example 7.

FIG. 10B is a chemiluminescence photograph of the western blot analysis on the effect of apoEdp on VEGF-induced phosphorylation of eNOS, as explained in Example 7.

FIG. 11A is a graphical representation of the effect of apoEdp on VEGF-induced phosphorylation of FAK, as explained in Example 7.

FIG. 11B is a chemiluminescence photograph of the western blot analysis of the effect of apoEdp on VEGF-induced phosphorylation of FAK, as explained in Example 7.

FIG. 12A is a graphical representation of the effect of apoEdp on VEGF-induced phosphorylation of Erk1/2, as explained in Example 7.

FIG. 12B is a chemiluminescence photograph of the western blot analysis of the effect of apoEdp on VEGF-induced phosphorylation of Erk1/2, as explained in Example 7.

### DISCLOSURE

An embodiment of the present invention relates to stopping tumor growth in mammals through the inhibition of angiogenesis by the introduction of an effective amount of apoEdp. ApoEdp's can inhibit angiogenesis by acting on cells involved in angiogenesis, endothelial cells. By blocking the activity of vascular endothelial growth factor induced signaling pathways, apoEdp can inhibit endothelial cell activity such as wound healing migration, capillary tube formation, and migration/invasion.

### EXAMPLES

### Example 1

### The Effect of ApoEdp on Cell Viability

To determine whether apoEdp affects cell survival, cell viability of human umbilical vein endothelial cells (HUVECs) and MDA-MB-231 human breast cancer cells was determined by using CellTiter 96® AQuous One solution cell proliferation kit (Promega, Madison, Wis.).

### Cells and Peptides

HUVECs were obtained from Lonza (Walkersville, Md.) and were maintained in endothelial cell basal medium (EBM-2) (Lonza, Walkersville, Md.) supplemented with 10% fetal bovine serum (FBS) and growth factors. (bullet kit, Lonza, Walkersville, Md.) HUVECs were used between passages 2-6. Human Breast Cancer cells MDA-MB-231 were obtained from the American Type Tissue Collection and cultured in Dulbecco's Modified Eagle's medium supplemented with 10% fetal bovine serum, 0.1 mg/mL streptomycin and 25 U/mL penicillin.

The 18 amino acid tandem repeat dimer peptide of apolipoprotein E was synthesized with a purity of greater than 95% (Genemed, Arlington, Tex.) and was derived from the human apolipoprotein E receptor binding region between residues 141 and 149.

### Procedure

The HUVECs, as described above, were seeded in 96-well plates at about 10,000 cells per well. The next day, the cells were starved for 18 hours in EBM-2+1% FBS without any growth supplement. The HUVECs were then incubated with apoEdp in various concentrations in the range of about 6 μM to about 300 μM. An hour later, 50 ng/mL of vascular endothelial growth factor (VEGF) was introduced. The cells were incubated for two days, and cell numbers were determined using the CellTiter96 AQuous One Solution cell proliferation kit. (Promega, Madison, Wis.)

The MDA-MB-231 cells were in 96-well plates at about 5,000 cells per well. The MDA-MB-231 cells were then allowed to adhere to the plate with apoEdp in various concentrations in the range of about 6 μM to about 300 μM. The cells were incubated for two days, and cell numbers were determined using the CellTiter 96® AQuous One Solution cell proliferation kit. (Promega, Madison, Wis.)

### Results

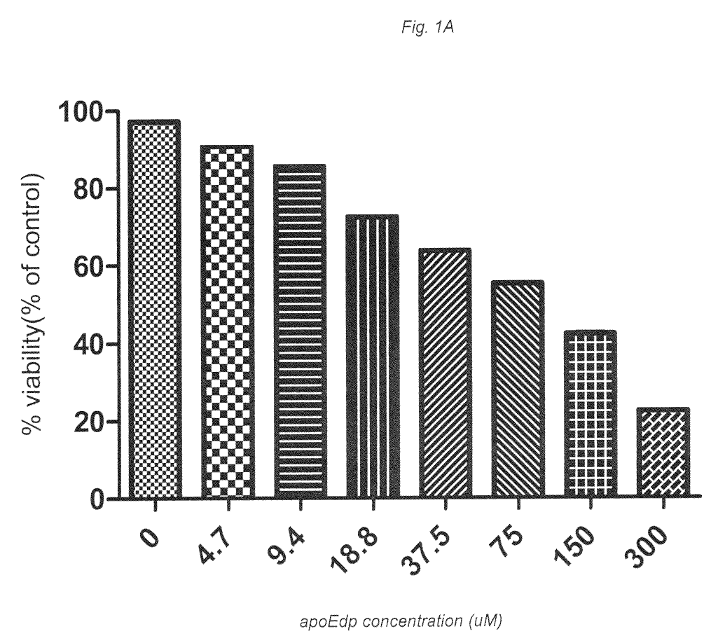

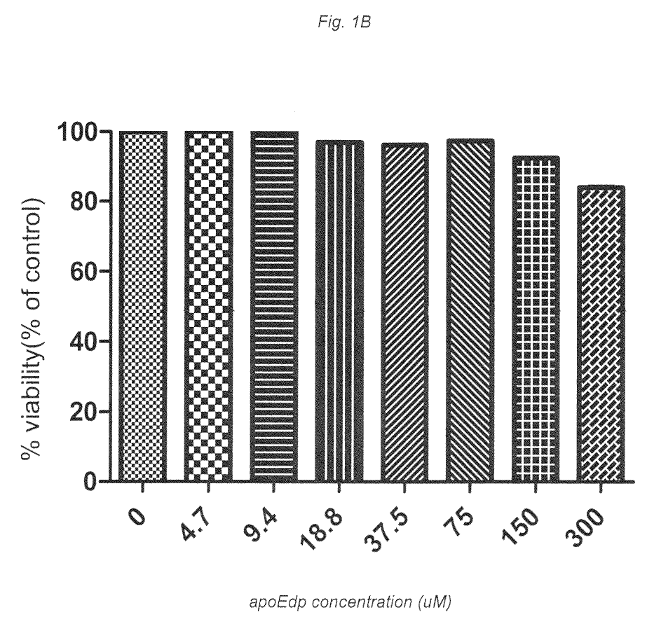

ApoEdp treatment of HUVECs induced a dose-dependent inhibition of cell viability. The 50% cytotoxic dose for the HUVECs as determined by the assay was about 103 μM. (See FIG. 1A) However, apoEdp treatment of MDA-MB-231 human breast cancer cells appeared to have no effect on cell viability. The 50% cytotoxic dose for the MDA-MB-231 human breast cancer cells was something higher than the upper limit of apoEdp concentration tested by the experiment, 300 μM. (See FIG. 1B)

### Example 2

### The Effect of ApoEdp on Endothelial Cell Wound Healing Migration

The effect of apoEdp on VEGF-induced endothelial cell migration was tested by performing wound-healing assays utilizing HUVECs, as prepared in Example 1.

### Procedure

The HUVECs were allowed to grow to confluency on 6-well plates and washed twice with phosphate buffer solution (PBS). Monolayer cells were then wounded by scratching with a 1-mL pipette tip, washed three times with PBS, and incubated for eight hours in EBM-2 with 1% FBS without growth supplements. The cells were then supplemented with 1% FBS and 50 ng/mL of VEGF. There were three groups of cells: a group of cells without VEGF, a group with VEGF, and a group with VEGF and apoEdp. The experimental control group was established without apoEdp. The test group was given apoEdp in various concentrations in the range of about 6 μM to about 100 μM.

### Results

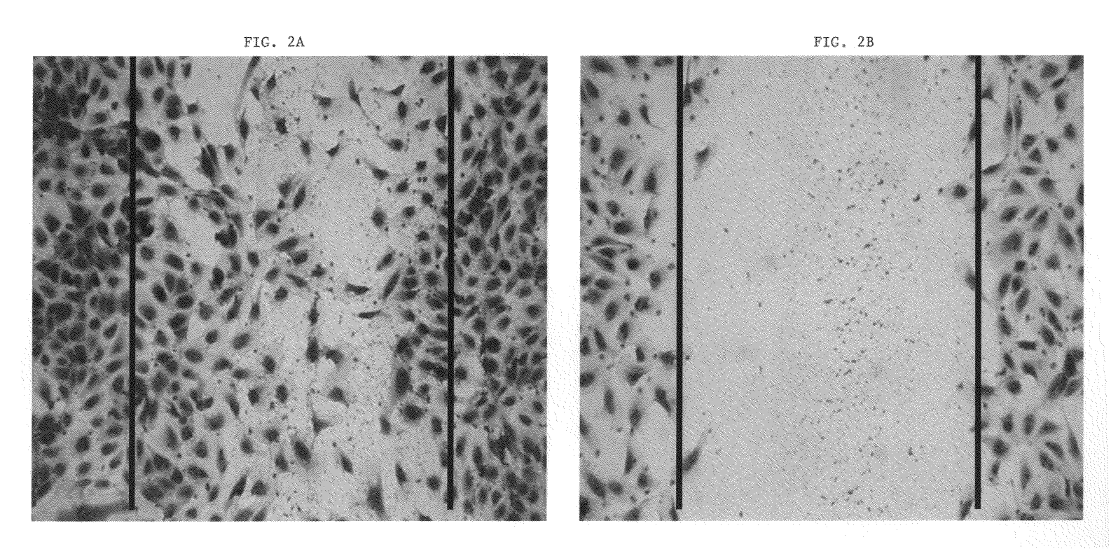

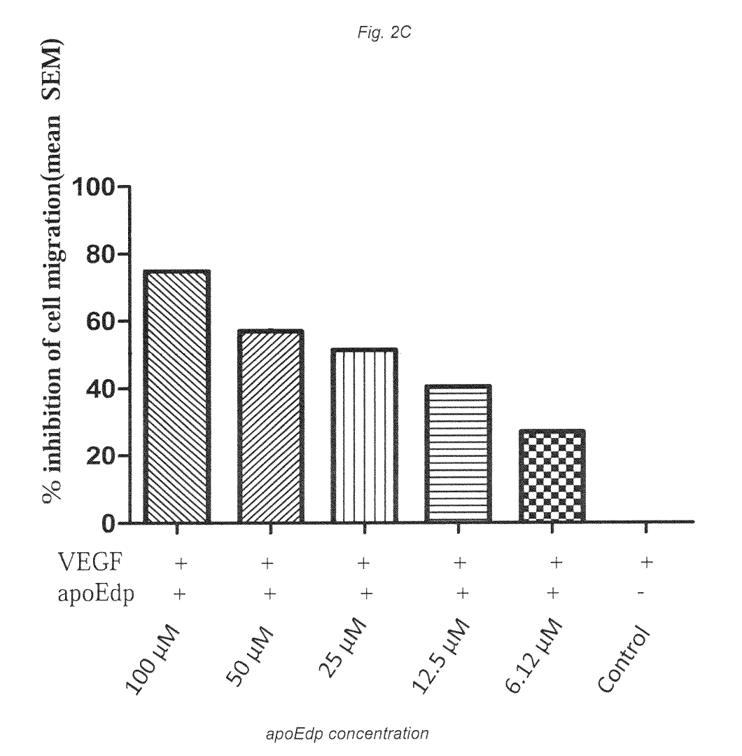

A wound healing assay was used to measure changes in cell migration. See FIGS. 2A and 2B. The results showed that the inhibitory effect of apoEdp on HUVEC wound-healing migration is dose dependent at the tested range of about 6 μM to about 100 μM. The 50% inhibitory concentration (IC₅₀) for the assays is about 21.5 μM. (See FIG. 2C)

### Example 3

### The Effect of ApoEdp on Endothelial Cell Matrigel Tubule Formation

The effect of apoEdp on VEGF-induced endothelial cell tubule formation was tested by microtubule formation assays of HUVECs, as prepared in Example 1,

### Procedure

Matrigel (Sigma Aldrich, St. Louis, Mo.) was thawed overnight on ice; each well of 24-well plates was then coated at 4 degrees Celsius with 300 μL matrigel and incubated at 37 degrees Celsius for thirty minutes. HUVECs, as described in Example 1, were harvested and about 4×10⁴ cells per well were plated. Microtubule formation was assessed after twelve to sixteen hours using an inverted photomicroscope. The images were photographed using an Olympus U-RLF-T microscope. The tubule structures were then counted manually and the percentage of inhibition of formation was expressed using the VEGF control wells as 100% formation.

### Results

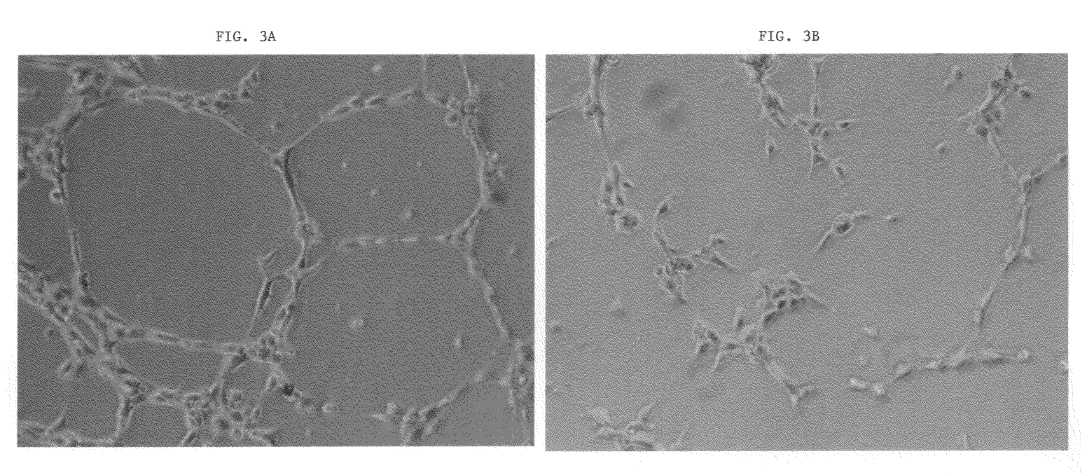

Upon quantitative analysis of the plates, it was found that apoEdp significantly inhibited the ability of HUVECs to form capillary tubes when compared to the VEGF control. See FIGS. 3A and 3B. ApoEdp's inhibition of microtubule formation was concentration dependent in the range of about 6 μM to about 50 μM. The 50% inhibitory concentration (IC₅₀) for the assays was about 9 μM. (See FIG. 3C)

### Example 4

### The Effect of ApoEdp on Endothelial Cell Migration/Invasion

The effect of apoEdp on VEGF-induced endothelial cell invasion/migration was tested by performing invasion/migration assays utilizing HUVECs, as prepared in Example 1.

### Procedure

Boyden chamber migration/invasion assays were performed using 24-well transwell (BD Biosciences, San Jose, Calif.) migration chambers with an 8 μm pore size. The transwells were then placed in the 48-well plate where the bottom chambers were filled with 600 μL medium. The medium in the chambers for the test group were supplemented with VEGF while the medium in the chambers for the control group was not. The top chamber was then seeded with about 4×10⁴ cells per well of HUVECs in 100 μL containing various concentrations of apoEdp in the range of about 6 μM to about 100 μM.

The HUVECs were then allowed to migrate for sixteen hours at thirty seven degrees Celsius. On the top surface, the HUVECs were gently scraped with cotton swabs. On the bottom surface, the HUVECs were fixed with 10% buffered formalin for twenty minutes, washed three times with PBS, and stained with hematoxylin and eosin. Next, the HUVECs were de-stained in PBS, and the transwell membrane was allowed to dry at room temperature.

### Results

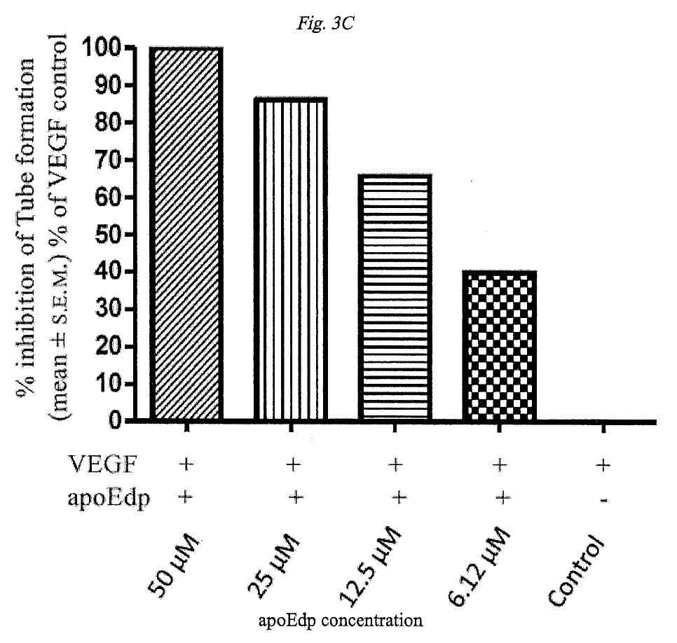

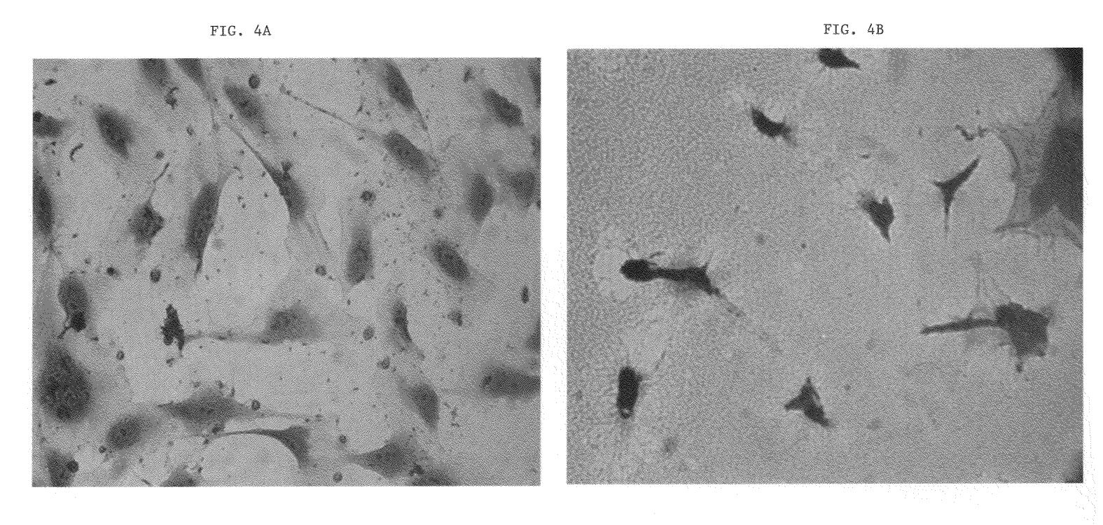

The transwell migrated/invaded cells were counted using an inverted microscope. Three independent areas per filter were counted, and the mean+/−SEM number of migrated/invaded cells was calculated. Compared to the VEGF control (See FIG. 4A), apoEdp significantly inhibited VEGF-induced cell migration/invasion. (See FIG. 4B) Inhibition of HUVEC migration/invasion was dose dependent in the range of about 6 μM to 100 μM. The 50% inhibitory concentration (IC₅₀) for the assays was about 31.5 μM. (See FIG. 4C)

### Example 5

### The Effect of ApoEdp on Angiogenesis in Rabbit Corneal Micropocket

### Assay

The effect of apoEdp on angiogenesis in vivo was observed by testing its inhibitory effect on VEGF-induced angiogenesis in a corneal micropocket assay of a rabbit eye model.

### Procedure

A corneal micropocket was created with a modified von Graefe cataract knife in each eye of several 2-3 pound New Zealand White Rabbits. A micropellet of 500 μm by 500 μm was prepared which contained 0.4 g of Compritol 888 ATO (Gattefosse) combined with 0.1 g of Squalane oil (Sigma Aldrich) and 20 mg/mL L-α-Phosphatidylcholine. The 20 mg/mL L-α-Phosphatidylcholine contained either saline for the control subjects or 160 ng of VEGF for the test subjects. The right eye of each test subject was implanted with a VEGF-containing pellet and the left eye of each test subject was implanted with a saline-containing pellet. Each of the pellets was positioned about 2 mm from the corneal limbus.

A group of five rabbits was treated with eye drops containing a 1% apoEdp solution and a second group of five rabbits was treated with eye drops containing a saline solution. Treatment began one day post-implantation (PI) and continued for five consecutive days. The drops had a volume of 50 μL and were applied topically five times per day with two hours separating the five dosages.

### Results

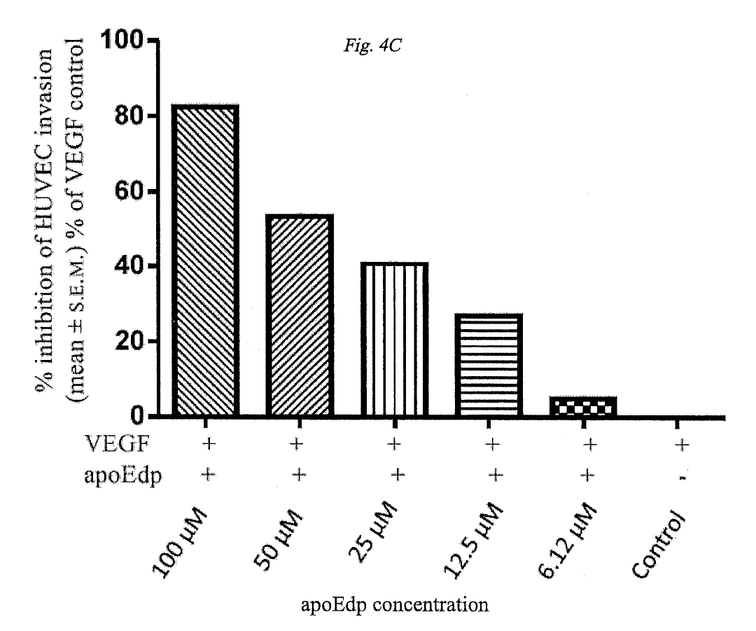

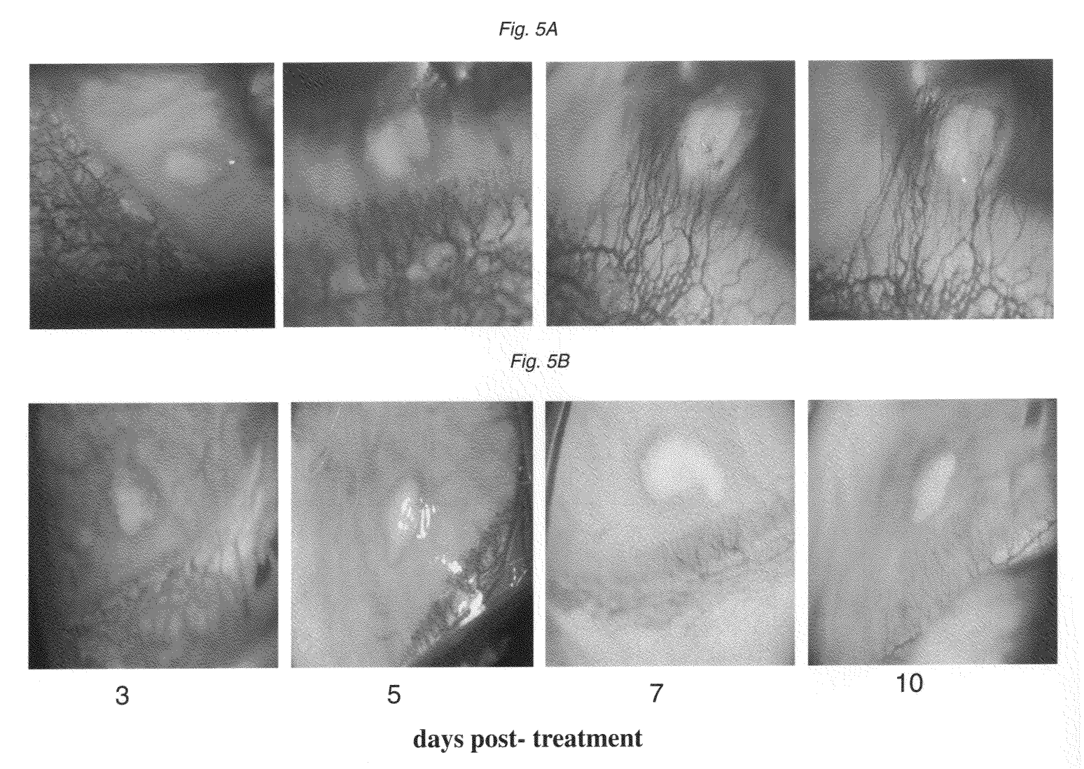

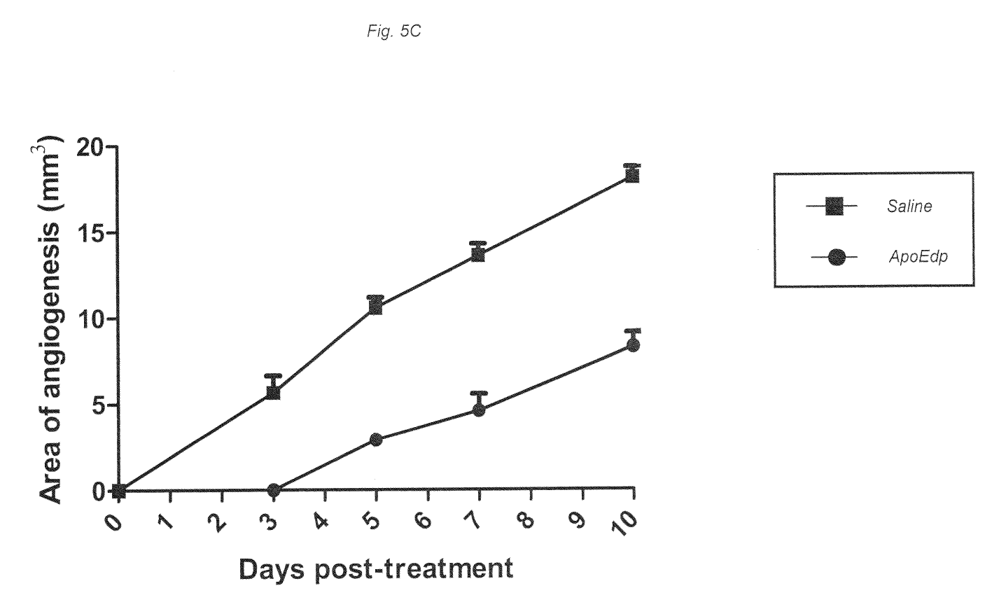

Data (See FIG. 5C) and photographs (See FIG. 5A and FIG. 5B) were obtained from Days 3-10 post-implantation. The area of neovascular response, vessel length and clock hours of new blood vessel formation of each group were all calculated according to the formula, Area (mm²)=C/12×3.1416[r²−(r−L)²] where, C=the number of clock hours at the limbus involved in the neovascular response, L=the length of the longest neovascular pedicle from the limbus onto the anterior cornea, and r=the radius of the cornea.

Topical application of the eye drops containing 1% apoEdp in the test subjects (See FIG. 5A) significantly inhibited VEGF-induced angiogenesis compared to topical application of the eye drops containing only saline in the same test subjects (See FIG. 5B). The graphical representation of the physical data is shown in FIG. 5C.

### Example 6

### The Effect of ApoEdp on Tumor Growth in Nude Mice

The effect of apoEdp on tumor growth in vivo was observed by testing its inhibitory effect on xenograft tumor growth post-injection in nude mice.

### Procedure

The test subjects were female nu/nu mice, aged 8-12 weeks, weighing about 20 g. (Charles River Laboratories, Harlan, Indianapolis, Ind.) The mice were weighed, coded, and divided into experimental groups at random. The mice were subcutaneously injected with about 3×10⁶ MDA-MB-231 human breast cancer cells in 100 μL PBS. The cells were injected into the right sides of the dorsal area of the test subjects. The tumor size was allowed to reach about 100 mm³ which took about ten days.

Following injection of the tumor cells, the subjects were given injections for three consecutive days. About 40 mg/kg/day of apoEdp was injected intralesionally into the test subjects, and about 100 μA of sterile PBS was injected intralesionally into the control subjects.

### Results

In order to evaluate tumor growth, tumor volume was determined every seven days by measuring the tumor with a digital caliper and calculating tumor volume. Volume was calculated using the formula, V=A×B²×0.52, where A=longest diameter of the tumor, and B=the shortest diameter of the tumor.

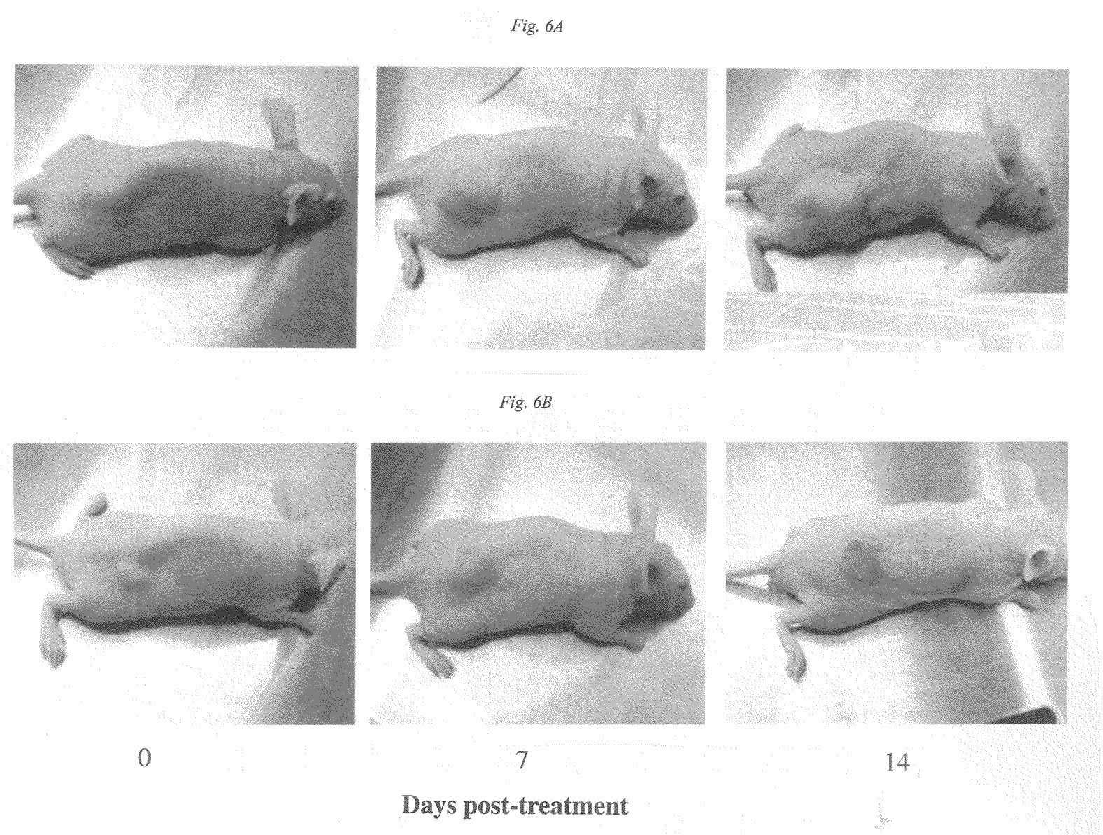

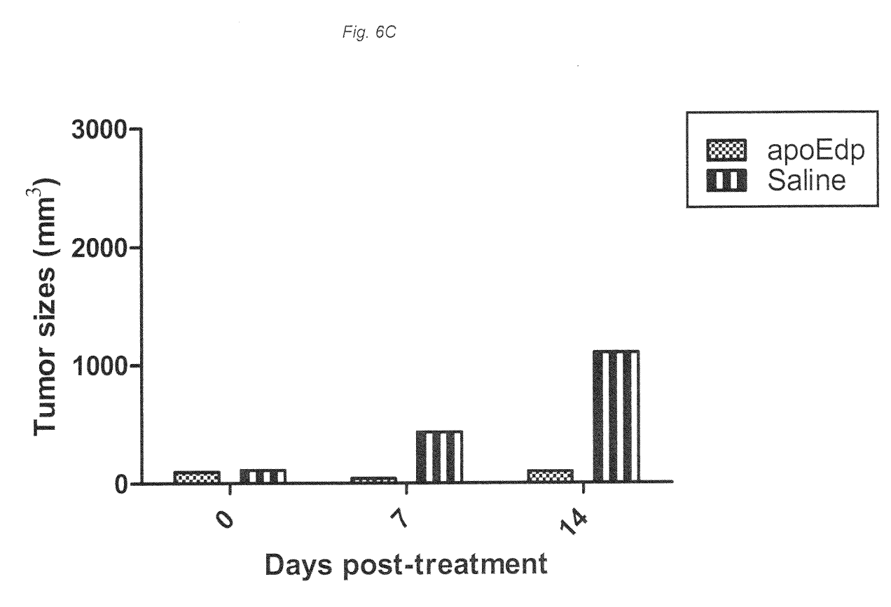

Test subjects treated with apoEdp demonstrated that apoEdp significantly inhibited tumor growth (See FIG. 6A) compared to the control subjects treated with PBS. (See FIG. 6B) Additionally, there was no significant body weight difference between the test group treated with apoEdp and the control group treated with PBS. No observable signs of toxicity of apoEdp were detected during treatment.

### Example 7

### The Effect of ApoEdp on VEGF-Induced Signaling Pathways

The mechanism by which apoEdp inhibits angiogenesis and tumor growth was evaluated using Western Blot Analysis.

### Procedure

Confluent HUVECs were grown in EBM-2 containing 1% FBS for twenty-four hours. The medium was then replaced with 1% FBS media in the presence or absence of apoEdp in the range of about 6 μM to about 50 μM. The cells were allowed to grow for one hour; then VEGF was added at a concentration of 10 ng/mL. The cells were then incubated for ten minutes to detect the phosphorylated forms of angiogenic signaling molecules.

The cells were lysed, quantified for protein concentration, and separated on 4% to 20% pre-cast SDS-PAGE gels. Western blots were then performed of the control cell lysates and the apoEdp treated cell lysates. The western blots were performed with antibodies against the phosphorylated and non-phosphorylated (control) forms of Flk-1, c-Src, FAK, Erk1/2, Akt and eNOS. (Santa Cruz Biotechnology, Santa Cruz, Calif.).

### Results

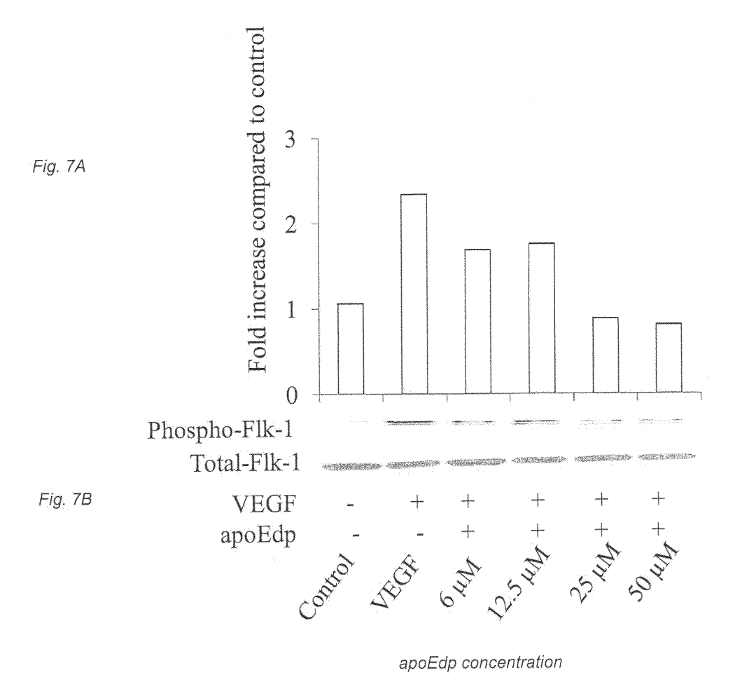

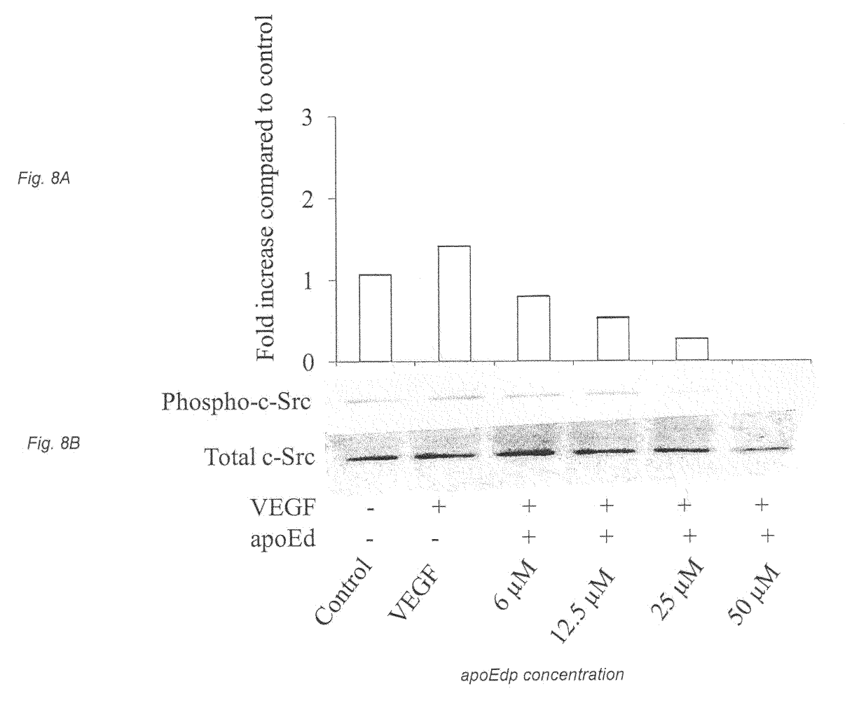

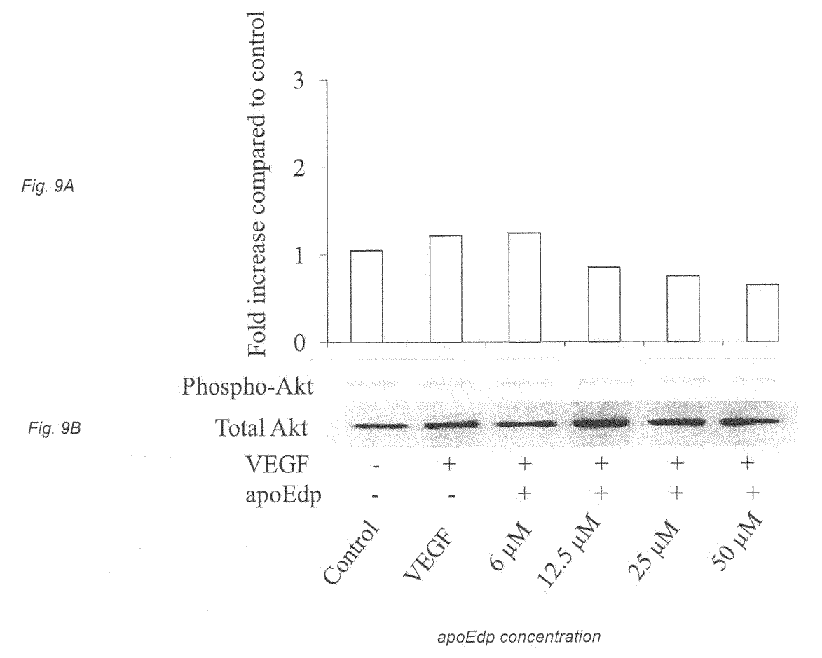

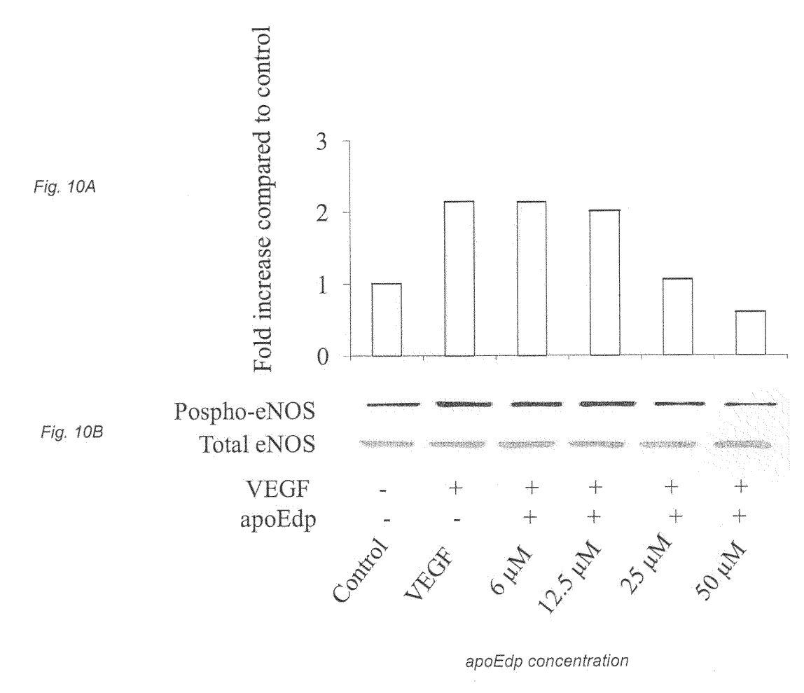

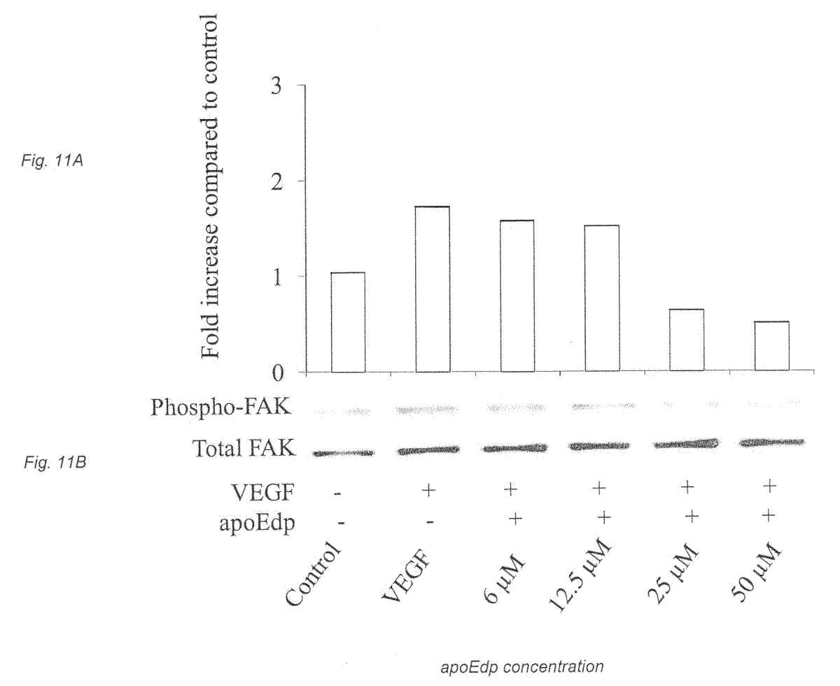

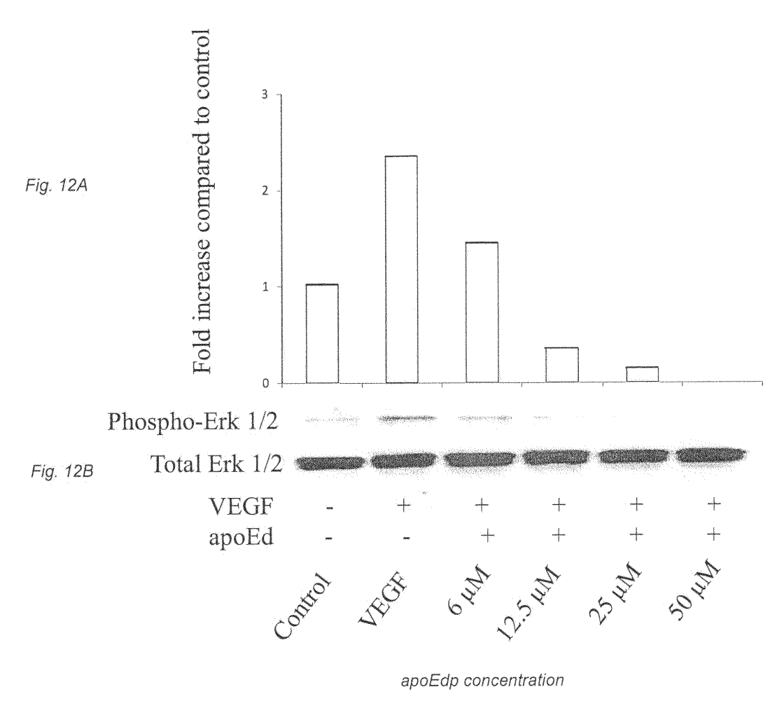

Experimental results indicated that apoEdp significantly inhibited VEGF-induced phosphorylation of Flk-1 (See FIG. 7A), c-Src (See FIG. 7B), Akt (See FIG. 7C), eNOS (See FIG. 7D), FAK (See FIG. 7E), and Erk1/2 (See FIG. 7F) in a concentration dependent manner.

1. A method to inhibit tumor growth in mammals, said method comprising introduction of an 18 amino acid tandem-repeat dimer peptide of apolipoprotein E residues 141-149 into a mammal, in an amount effective to inhibit tumor growth in said mammal compared to the rate of tumor growth which would occur in the absence of said apolipoprotein E dimer peptide, whereby said tumor growth is inhibited.

2. The method of claim 1 whereby said introduction further comprises at least one injection.

3. The method of claim 1 whereby said introduction further comprises at least one topical application.

4. The method of claim 2 whereby said injection amount is from about 10 mg/kg/day to about 80 miligrams/kilogram/day.

5. The method of claim 2 whereby said injection amount is about 40 miligrams/kilogram/day.

6. The method of claim 3 whereby said application amount of said 18 amino acid tandem-repeat dimer peptide of apolipoprotein E is from about 0.1 percent (weight/volume) to about 4 percent (weight/volume).

7. The method of claim 3 whereby said application amount of said 18 amino acid tandem-repeat dimer peptide of apolipoprotein E is from about 1 percent (weight/volume) to about 3 percent (weight/volume).

8. The method of claim 3 whereby said application amount of said 18 amino acid tandem-repeat dimer peptide of apolipoprotein E is about 1 percent (weight/volume).

9. A method for inhibiting endothelial cell proliferation, said method comprising application of an 18 amino acid tandem-repeat dimer peptide of apolipoprotein E residues 141-149, whereby said application amount is effective to decrease endothelial cell proliferation compared to endothelial cell proliferation which would occur in the absence of said apolipoprotein E dimer peptide, whereby said endothelial cell proliferation is decreased.

10. The method of claim 9 whereby said effective amount of said 18 amino acid tandem repeat dimer peptide of apolipoprotein E is between about 4.7 to about 300 micromoles per liter of solvent.

11. The method of claim 9 whereby said effective amount of said 18 amino acid tandem repeat dimer peptide of apolipoprotein E is about 103 micromoles per liter of solvent.

12. A method for inhibiting endothelial cell wound healing migration, said method comprising application of an 18 amino acid tandem-repeat dimer peptide of apolipoprotein E residues 141-149, whereby said application amount is effective to decrease endothelial cell wound healing migration compared to endothelial cell wound healing migration which would occur in the absence of apolipoprotein E dimer peptide, whereby said endothelial cell wound healing migration is decreased.

13. The method of claim 12 whereby said effective amount of said 18 amino acid tandem repeat dimer peptide of apolipoprotein E is between about 6 to about 100 micromoles per liter of solvent.

14. The method of claim 12 whereby said effective amount of said 18 amino acid tandem repeat dimer peptide of apolipoprotein E is about 21.5 micromoles per liter of solvent.

15. A method for inhibiting endothelial cell capillary tube formation, said method comprising application of an 18 amino acid tandem-repeat dimer peptide of apolipoprotein E residues 141-149, whereby said application amount is effective to decrease endothelial cell capillary tube formation compared to endothelial cell capillary tube formation which would occur in the absence of said 18 amino acid tandem-repeat dimer peptide of apolipoprotein E residues 141-49, whereby said endothelial cell capillary tube formation is decreased.

16. The method of claim 15 whereby said effective amount of said 18 amino acid tandem repeat dimer peptide of apolipoprotein E is between about 6 to about 50 micromoles per liter of solvent.

17. The method of claim 15 whereby said effective amount of said 18 amino acid tandem repeat dimer peptide of apolipoprotein E is about 9 micromoles per liter of solvent.

18. A method for inhibiting endothelial cell invasion, said method comprising application of an 18 amino acid tandem-repeat dimer peptide of apolipoprotein E residues 141-149, whereby said application amount is effective to decrease endothelial cell invasion compared to endothelial cell invasion which would occur in the absence of said 18 amino acid tandem-repeat dimer peptide of apolipoprotein E residues 141-49, whereby said endothelial cell invasion is decreased.

19. The method of claim 18 whereby said effective amount of said 18 amino acid tandem repeat dimer peptide of apolipoprotein E is between about 6 to about 100 micromoles per liter of solvent.

20. The method of claim 18 whereby said effective amount of said 18 amino acid tandem repeat dimer peptide of apolipoprotein E is about 31.5 micromoles per liter of solvent.

21. A method for interrupting the activity of a vascular endothelial growth factor-mediated signaling pathway, said method comprising introduction of an 18 amino acid tandem-repeat dimer peptide of apolipoprotein E residues 141-149 in an amount effective to interrupt the activity of said vascular endothelial growth factor-mediated signaling pathway compared to the activity of said vascular endothelial growth factor-mediated signaling pathway which would occur in the absence of said 18 amino acid tandem-repeat dimer peptide of apolipoprotein E residues 141-149, whereby said introduction interrupts a vascular endothelial growth factor-induced signaling pathway.

22. The method of claim 21 whereby said signaling pathway is at least one signaling pathway selected from the group consisting of Flk-1, c-Src, Akt, eNOS, FAK, and Erk 1/2.
